(19) United States
(12) Patent Application Publication
(10) Pub. No.: US 2022/0257465 A1
(43) Pub. Date: Aug. 18, 2022

(54) CARDIO PULMONARY RESUSCITATION FEEDBACK SYSTEM

(71) Applicant: HeartSine Technologies Limited, Belfast (IE)

(72) Inventors: Dymphna Mary DONAGHY, Co. Donegal (IE); Johnny Houston ANDERSON, Holywood (GB); Olibhear Oisin Gerard McALISTER, Belfast (GB); Adam Patrick HARVEY, Hillsborough (GB)

(21) Appl. No.: 17/674,066

(22) Filed: Feb. 17, 2022

(30) Foreign Application Priority Data
Feb. 17, 2021 (EP) 21157655.08

(51) Int. Cl. A61H 31/00; A61N 1/39

(52) U.S. Cl. CPC A61H 31/005; A61N 1/39044; A61H 2230/045; A61H 2230/405; A61H 2230/208; A61H 2230/655

(57) ABSTRACT

A CPR feedback system for assessing CPR carried out by a person on a subject and providing CPR feedback to the person, including an ECG system, a biosignal system, a CPR assessment system, and a CPR feedback unit. The CPR assessment system is configured to perform the steps (i) establish a reference ECG signal metric and a target biosignal metric, (ii) produce a CPR feedback signal, (iii) receive ECG signals measured during a plurality of chest compressions and use the ECG signals to establish a current ECG signal metric, (iv) receive biosignals and use the biosignals to establish a current biosignal metric, (v) compare the current ECG signal metric with the reference ECG signal metric and compare the current biosignal metric with the target biosignal metric, (vi) when the current ECG signal metric is less than the reference ECG signal metric and the current biosignal metric is less than the target biosignal metric, produce a CPR feedback signal, (vii) when the current ECG signal metric is less than the reference ECG signal metric and the current biosignal metric is equal to or greater than the target biosignal metric, increase the target biosignal metric and produce a CPR feedback signal advising the person to improve CPR performance, (viii) when the current ECG signal metric is equal to or greater than the reference ECG signal metric, set the reference ECG signal metric equal to the current ECG signal metric and produce a CPR feedback signal advising the person to maintain current CPR performance.

### FIELD OF THE INVENTION

This disclosure relates to a cardio pulmonary resuscitation (CPR) feedback system, using electrocardiogram (ECG) signals and biosignals to assess the CPR and provide CPR feedback to a person during their performance of CPR on a subject.

### BACKGROUND

CPR involves the compression of the chest of the subject to cause the heart to pump blood around the circulatory system, primarily to provide oxygenated blood to the subject's heart and brain. If the compression of the chest is too shallow, too slow or too fast, then the heart will not pump sufficient oxygenated blood.

### SUMMARY

The CPR feedback system is used to treat a subject by instructing a person using the system to commence and maintain high quality CPR. Changes in chest compression rate and chest compression depth evoke changes in biosignals measured from the subject. These biosignals may be used to guide the person on CPR performance. It is advantageous if CPR feedback is provided to the person during CPR, as this can improve the CPR performance of the person.

According to a first aspect of the disclosure there is provided a cardio pulmonary resuscitation (CPR) feedback system for assessing CPR carried out by a person on a subject and providing CPR feedback to the person, including one or more of:

an electrocardiogram (ECG) system configured to measure ECG signals of the subject,

a biosignal system configured to measure biosignals of the subject,

a CPR assessment system connected to the ECG system to receive ECG signals and connected to the biosignal system to receive biosignals, and

a CPR feedback unit connected to the CPR assessment system and configured to receive CPR feedback signals and issue CPR feedback to the person,

wherein the CPR assessment system is configured to perform one or more of the steps: (i) establish a reference ECG signal metric and a target biosignal metric, (ii) produce a CPR feedback signal advising the person to start CPR, (iii) receive ECG signals measured during a plurality of chest compressions and use the ECG signals to establish a current ECG signal metric, (iv) receive biosignals measured during the plurality of chest compressions and use the biosignals to establish a current biosignal metric, (v) compare the current ECG signal metric with the reference ECG signal metric and compare the current biosignal metric with the target biosignal metric, (vi) when the current ECG signal metric is less than the reference ECG signal metric and the current biosignal metric is less than the target biosignal metric, produce a CPR feedback signal advising the person to improve CPR performance, (vii) when the current ECG signal metric is less than the reference ECG signal metric and the current biosignal metric is equal to or greater than the target biosignal metric, increase the target biosignal metric and produce a CPR feedback signal advising the person to improve CPR performance, and/or (viii) when the current ECG signal metric is equal to or greater than the reference ECG signal metric, set the reference ECG signal metric equal to the current ECG signal metric and produce a CPR feedback signal advising the person to maintain current CPR performance.

The CPR feedback system provides dynamic CPR feedback to the person performing CPR on the subject, the CPR feedback being based on repeated monitoring of the chest compressions performed by the person on the subject using biosignal measurements and repeated monitoring of the ECG of the subject.

The biosignal system may be configured to measure one or more type of biosignals of the subject. The types of biosignals may comprise any of chest impedance signals, end-tidal carbon dioxide signals, saturation of peripheral oxygen signals, blood pressure signals, chest compression depth signals. The biosignal system may comprise any of an impedance signal measurement system, a capnograph, an oximeter, a blood pressure measurement system, an accelerometer.

Establishing the reference ECG signal metric may comprise receiving ECG signals from the ECG system measured over a predefined period of time prior to commencement of CPR and using the ECG signals to establish the reference ECG signal metric.

Using the ECG signals to establish the reference ECG signal metric may comprise establishing a score of the ECG signals. The score may relate to quality of the measured ECG signals. The score may be derived from one or more time-domain features of the ECG signals. The time-domain features may comprise any of mean amplitude, peak amplitude, median slope of the ECG signals. The score may be derived from one or more frequency-domain features of the ECG signals. The frequency-domain features may comprise any of amplitude spectrum area (AMSA), power spectrum analysis features, centroid frequency. The score may be derived from one or more time-domain features and one or more frequency-domain features. The time-domain features and the frequency-domain features may be used as measures to estimate a condition of the subject.

Establishing the target biosignal metric may comprise receiving a pre-determined target biosignal metric. The pre-determined target biosignal metric may comprise at least one target biosignal metric component for one or more types of biosignal. The at least one target biosignal metric component for the one or more types of biosignal may comprise any of at least one target frequency biosignal metric component, at least one target amplitude biosignal metric component, at least one target frequency biosignal metric component and at least one target amplitude biosignal metric component.

Establishing the target biosignal metric may comprise (i) receiving one or more types of biosignals measured during a plurality of chest compressions by the person, and (ii) using the or each or some of the types of biosignals to establish the target biosignal metric. The target biosignal metric may comprise at least one target biosignal metric component for the or each or some of the types of biosignal. The at least one target biosignal metric component for the or each or some of the types of biosignal may comprise any of at least one target frequency biosignal metric component, at least one target amplitude biosignal metric component at least one target frequency biosignal metric component and at least one target amplitude biosignal metric component. The target biosignal metric component for each type of biosignals may provide a measure of target CPR performance for the person during CPR chest compressions.

The target amplitude biosignal metric components may comprise any of average amplitude, average local maxima, average local minima, ratio of local maxima to local minima. The target frequency biosignal metric component may comprise dominant frequency.

Receiving ECG signals measured during a plurality of chest compressions may comprise receiving ECG signals measured over a window of predefined duration. The predefined duration may be in a range of approximately 5 seconds to approximately 30 seconds.

Using the ECG signals to establish a current ECG signal metric may comprise establishing a score of the ECG signals. The score may relate to quality of the measured ECG signals. The score may be derived from one or more time-domain feature of the ECG signals. The time-domain features may comprise any of mean amplitude, peak amplitude, median slope of the ECG signals. The score may be derived from one or more frequency-domain features of the ECG signals. The frequency-domain features may comprise any of amplitude spectrum area (AMSA), power spectrum analysis features, centroid frequency. The score may be derived from one or more time-domain features and one or more frequency-domain features. The time-domain features and the frequency-domain features may be used as measures to estimate subject condition.

The ECG signals may be assessed to confirm the presence of ventricular fibrillation (VF) signals.

Receiving biosignals measured during the plurality of chest compressions may comprise receiving biosignals measured over a window of predefined duration. The predefined duration may be in a range of approximately 5 seconds to approximately 30 seconds.

Receiving biosignals measured during the plurality of chest compressions may comprise receiving at least one type of biosignals measured during the plurality of chest compressions. Using the biosignals measured during the plurality of chest compressions to establish the current biosignal metric may comprise establishing at least one current biosignal metric component for at least one type of biosignals. The at least one current biosignal metric component for the at least one type of biosignals may comprise any of at least one current frequency biosignal metric component, at least one current amplitude biosignal metric component, at least one current frequency biosignal metric component and at least one current amplitude biosignal metric component. The current biosignal metric component for the least one type of biosignals may provide a measure of actual CPR performance by the person during the plurality of CPR chest compressions.

The current amplitude biosignal metric components may comprise any of average amplitude, average local maxima, average local minima, ratio of local maxima to local minima. The current frequency biosignal metric component may comprise dominant frequency.

Comparing the current biosignal metric with the target biosignal metric may comprise comparing at least one current biosignal metric component for at least one type of biosignals with at least one equivalent target biosignal metric component for the at least one type of biosignals.

Increasing the target biosignal metric may comprise increasing the metric by 1% to 50%.

The CPR feedback signal advising the person on how to adjust CPR may be received by the feedback unit and cause the feedback unit to issue CPR feedback to the person in the form of ‘Push Harder’ and/or ‘Push Faster’. The CPR feedback signal advising the person to maintain current CPR performance may be received by the feedback unit and cause the feedback unit to issue CPR feedback to the person in the form of ‘Good Compressions’.

The CPR assessment system may be configured to repeat steps (iii) to (viii) over a predetermined period of time. The pre-determined period of time may be 2 minutes. When the predetermined period of time has been reached, the CPR assessment system may be configured to produce a CPR feedback signal advising the person to stop CPR. The CPR assessment system may then be configured to repeat steps (i) to (viii).

The CPR assessment system may be further configured to carry out the steps:

produce a CPR feedback signal advising the person to perform CPR chest compressions at a rate equal to a rate of an audible metronome signal emitted by a metronome of the CPR assessment system,

receive ECG signals measured during the chest compressions and use the ECG signals to establish a current ECG signal metric,

compare the current ECG signal metric with the reference ECG signal metric,

when the current ECG signal metric is less than the reference ECG signal metric, adjust the rate of the audible metronome signal, produce a CPR feedback signal advising the person to adjust the rate of performance of the CPR chest compressions to equal the adjusted rate of the audible metronome signal and go back to the second step,

when the current ECG signal metric is equal to or greater than the reference ECG signal metric, produce a CPR feedback signal advising the person to maintain the rate of performance of the CPR chest compressions.

The CPR feedback signal advising the person to adjust the rate of performance of the CPR chest compressions to equal the adjusted rate of the audible metronome signal may be received by the feedback unit and cause the feedback unit to issue CPR feedback to the person in the form of ‘Push Faster’ or ‘Push Slower’. The CPR feedback signal advising the person to maintain the rate of performance of the CPR chest compressions may be received by the feedback unit and cause the feedback unit to issue CPR feedback to the person in the form of ‘Push Harder’ or ‘Good Compressions’.

The ECG system may be configured to measure ECG signals of the subject using signals received from one or more electrodes placed on the subject.

The biosignal system may be configured to measure biosignals of the subject using signals received from one or more sensors placed on the subject.

The CPR feedback system may be a stand-alone system. The CPR feedback system may be part of a further system. The further system may be a defibrillator.

When the CPR feedback system is part of a further system, a CPR start signal may be received from the further system. When the CPR feedback system is part of a further system, a CPR stop signal may be received from the further system.

According to a second aspect of the disclosure there is provided a defibrillator comprising a CPR feedback system according to the first aspect of the disclosure.

### BRIEF DESCRIPTION OF THE DRAWINGS

An embodiment of the disclosure will now be described, by way of example only, with reference to the following drawings, in which:

FIG. 1 is a schematic representation of a CPR feedback system according to the disclosure; and

FIG. 2 is a flowchart of steps carried out by a CPR assessment system of the CPR feedback system of FIG. 1.

### DETAILED DESCRIPTION

The following disclosure describes a number of different innovations related to a CPR feedback system, as well as other innovations. Various examples of the disclosure are described in detail below. While specific implementations are described, it should be understood that this is done for illustration purposes only. Other components and configurations may be used without parting from the spirit and scope of the disclosure. When specific method examples are discussed, the various steps of the method examples can be implemented in different orders, combinations, or permutations, including additional steps, or excluding specific steps.

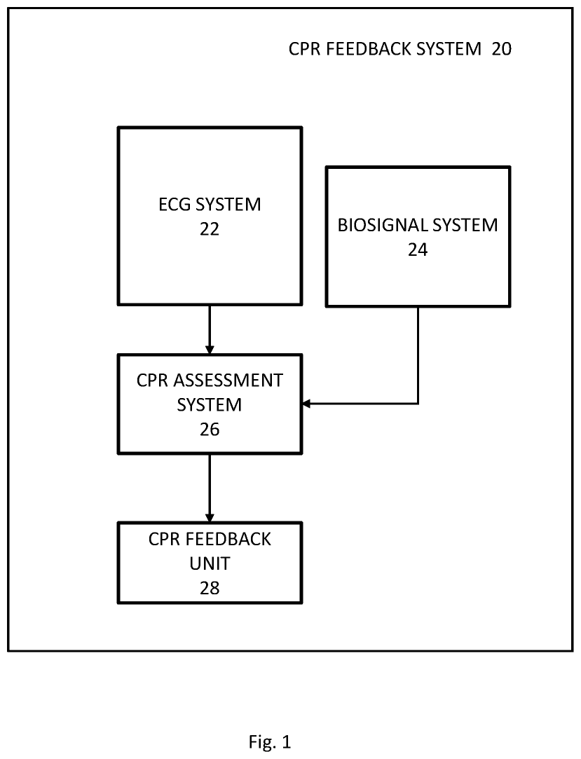

Referring to FIG. 1, the CPR feedback system 20 comprises an electrocardiogram (ECG) system 22, a biosignal system 24, a CPR assessment system 26 and a feedback unit 28. The CPR feedback system 20 assesses CPR carried out by a person (not shown) on a subject (not shown) and provides CPR feedback to the person. Th biosignal system 24 comprises each of an impedance signal measurement system, a capnograph, an oximeter, a blood pressure measurement system, an accelerometer.

It will be appreciated that the CPR feedback system 20 will comprise other elements such as an activation mechanism, an ECG processing system, a power source and a sensing unit which is adapted to be attached to the subject.

The ECG system 22 is connected to the sensing unit, which in this embodiment comprises thoracic electrodes, and is configured to measure ECG signals of the subject. The CPR feedback system 20 comprises an algorithm which uses the ECG signals to determine when the subject is exhibiting VF.

The impedance signal measurement system of the biosignal system 24 is connected to the sensing unit, which in this embodiment comprises thoracic electrodes, and measures impedance signals of the subject during chest compressions by the person. The impedance signals of the subject comprise thoracic impedance signals. The impedance signals includes sinusoidal waves, caused by the CPR chest compressions. The capnograph, oximeter, blood pressure measurement system and accelerometer of the biosignal system 24 are connected to sensors placed on the subject and receive biosignals of the subject from the sensors.

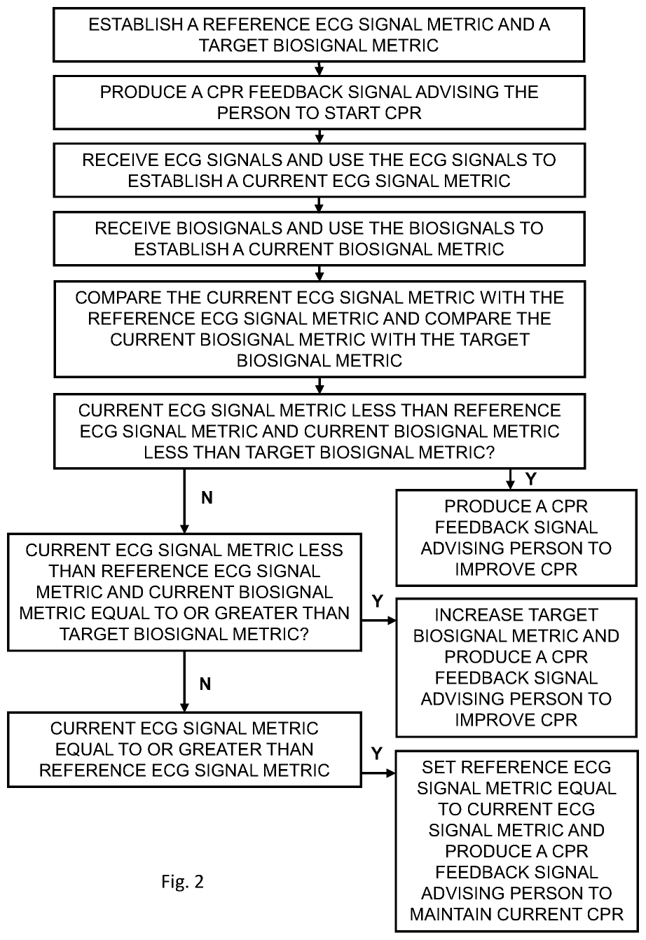

The CPR assessment system 26 is connected to the ECG system 22 and the biosignal system 24. The CPR assessment system 26 receives ECG signals and biosignals and assesses CPR performance by the person on the subject over multiple pluralities of chest compressions, delivered to the subject by the person in a pre-determined time. This includes performance of a number of steps, described below with reference to FIG. 2.

During assessment of the CPR performance of the person, the CPR assessment system 26 produces various feedback signals. The feedback unit 28 is connected to the CPR assessment system 26 and is configured to receive the feedback signals and issue CPR feedback to the person.

Referring to FIG. 2, a first embodiment of the steps performed by the CPR assessment system 26 of the CPR feedback system 20 of FIG. 1 will be described. In this embodiment, the biosignal system 24 uses its impedance measurement system to measure impedance signals of the subject. The CPR assessment system 26 receives one type of biosignals, i.e. impedance signals.

The CPR assessment system 26 first establishes a reference ECG metric. The CPR assessment system 26 receives ECG signals from the ECG system 22 measured over a predefined period of time of, for example, 5 to 30 seconds prior to commencement of CPR. The ECG signals are used to establish the reference ECG signal metric. This includes using the ECG signals to establish a reference ECG score of the ECG signals. The reference ECG score relates to quality of the measured ECG signals. In this embodiment, the reference ECG score is derived from one or more time-domain feature of the ECG signals and one or more frequency-domain features of the ECG signals. The time-domain features comprise any of mean amplitude, peak amplitude, median slope of the ECG signals. The frequency-domain features include any of amplitude spectrum area (AMSA), power spectrum analysis features, centroid frequency. The time-domain features and the frequency-domain features may be used as measures to estimate subject condition.

The CPR assessment system 26 then establishes a target biosignal metric. In this embodiment, establishing the target biosignal metric includes producing a CPR feedback signal advising the person to start CPR, receiving biosignals, comprising impedance signals, measured during a plurality of chest compressions by the person, producing a CPR feedback signal advising the person to stop CPR and using the biosignals to establish the target biosignal metric. In this embodiment, the target biosignal metric includes one target biosignal metric component for the type of biosignals measured, i.e. impedance signals. The target biosignal metric component includes a target amplitude biosignal metric component of average amplitude of peaks in the impedance signals measured during the plurality of CPR chest compressions by the person. It will be appreciated that the target biosignal metric may include more than one target biosignal metric component and that the components may be target amplitude biosignal metric components and/or target frequency biosignal metric components.

The CPR assessment system 26 then produces a CPR feedback signal advising the person to start CPR. This is sent to the feedback unit 28 which issues CPR feedback in the form of a ‘Start CPR and Push Hard’ instruction to the person using the CPR feedback system 20.

During a plurality of chest compressions by the person, the CPR assessment system 26 receives ECG signals from the ECG system 22, measured over a window of approximately 5 seconds to approximately 30 seconds. The CPR assessment system 26 uses the ECG signals to establish a current ECG signal metric, by establishing a current ECG score of the ECG signals. The current ECG score relates to quality of the measured ECG signals. The current ECG score is derived from one or more time-domain feature of the ECG signals and one or more frequency-domain features. The time-domain features include any of mean amplitude, peak amplitude, median slope of the ECG signals. The frequency-domain features include any of amplitude spectrum area (AMSA), power spectrum analysis features, centroid frequency. The time-domain features and the frequency-domain features may be used as measures to estimate subject condition.

During the plurality of chest compressions carried out by the person on the subject, the CPR assessment system 26 also receives impedance signals from the impedance measurement system of the biosignal system 24. The impedance signals are measured over the same window of approximately 5 seconds to approximately 30 seconds. The CPR assessment system 26 uses the impedance signals to establish a current biosignal metric. In this embodiment, the current biosignal metric includes one current biosignal metric component for the impedance signals. The current biosignal metric component includes a current amplitude biosignal metric component of average amplitude of peaks in the impedance signals measured during the window.

The CPR assessment system 26 compares the current ECG signal metric with the reference ECG signal metric and compares the current impedance signal metric with the target impedance signal metric. This includes comparing the reference ECG score with the current ECG score and comparing the target amplitude biosignal metric component of average amplitude of impedance signal peaks with the current amplitude biosignal metric component of average amplitude of impedance signal peaks. When the current ECG signal metric is less than the reference ECG signal metric (which suggests that the subject's condition is deteriorating) and the current biosignal metric is less than the target biosignal metric (which suggests that the CPR chest compressions are suboptimal), the CPR assessment system 26 produces a CPR feedback signal advising the person on how to adjust CPR. The feedback signal is sent to the feedback unit 28 which issues CPR feedback in the form of a ‘Push Harder’ instruction to the person using the CPR feedback system 20.

When the current ECG signal metric is less than the reference ECG signal metric (again suggesting that the subject's condition is deteriorating) and the current biosignal metric is equal to or greater than the target biosignal metric (which suggests that the target impedance signal metric may be improved), the CPR assessment system 26 increases the target biosignal metric and produces a CPR feedback signal advising the person to improve CPR performance. The feedback signal is sent to the feedback unit 28 which issues CPR feedback in the form of a ‘Push Harder’ instruction to the person using the CPR feedback system 20.

When the current ECG signal metric is equal to or greater than the reference ECG signal metric (suggesting that the subject's condition is improving), the CPR assessment system sets the reference ECG signal metric equal to the current ECG signal metric and produces a CPR feedback signal advising the person to maintain current CPR performance. The feedback signal is sent to the feedback unit 28 which issues CPR feedback in the form of a ‘Good Compressions’ instruction to the person using the CPR feedback system 20.

In this embodiment, the CPR assessment system 26 is configured to repeat some of the steps described above over a predetermined period of time. The pre-determined period of time is 2 minutes and is measured by a clock of the CPR assessment system 26. When the clock has not reached the pre-determined time the CPR assessment system 26 returns to the step of receiving ECG signals measured during a further plurality of chest compressions by the person. The further plurality of chest compressions do not overlap with the previous plurality of chest compressions. The analysis of the ECG and the biosignals comprising impedance signals and feedback to the subject therefore continues over several pluralities of chest compressions during the 2 min time. When the clock reaches the pre-determined time, the CPR assessment system 26 produces a CPR feedback signal advising the person to stop CPR. The feedback signal is sent to the feedback unit 28 which issues CPR feedback in the form of a ‘Stop CPR’ instruction to the person using the CPR feedback system 20.

In this embodiment, the CPR assessment system 26 then returns to the steps of establishing a reference ECG signal metric and a target biosignal metric and repeats the further steps as described above. This continues for as long as is deemed necessary.

Referring to FIG. 2, a second embodiment of the steps performed by the CPR assessment system 26 of the CPR feedback system 20 of FIG. 1 will be described. In this embodiment, the biosignal system 24 uses its oximeter and accelerometer to measure saturation of peripheral oxygen signals and CPR chest compression depth signals of the subject. The CPR assessment system 26 receives two types of biosignals, i.e. oxygen signals and CPR chest compression depth signals.

The CPR assessment system 26 first establishes a reference ECG metric. The CPR assessment system 26 receives ECG signals from the ECG system 22 measured over a predefined period of time of, for example, 5 to 30 seconds prior to commencement of CPR. The ECG signals are used to establish the reference ECG signal metric. This includes using the ECG signals to establish a reference ECG score of the ECG signals. The reference ECG score relates to quality of the measured ECG signals. In this embodiment, the reference ECG score is derived from one or more time-domain feature of the ECG signals and one or more frequency-domain features of the ECG signals. The time-domain features include any of mean amplitude, peak amplitude, median slope of the ECG signals. The frequency-domain features include any of amplitude spectrum area (AMSA), power spectrum analysis features, centroid frequency. The time-domain features and the frequency-domain features may be used as measures to estimate subject condition.

The CPR assessment system 26 then establishes a target biosignal metric. In this embodiment, establishing the target biosignal metric includes receiving a pre-determined target biosignal metric. The pre-determined target biosignal metric includes at least one target biosignal metric component for each type of biosignals measured. In this embodiment, the target biosignal metric components include a target amplitude biosignal metric component of average amplitude of oxygen signals and a target amplitude biosignal metric component of average CPR chest compression depth signals. It will be appreciated that the target biosignal metric may include more than one target biosignal metric component and that the components may be target amplitude biosignal metric components and/or target frequency biosignal metric components.

The CPR assessment system 26 then produces a CPR feedback signal advising the person to start CPR. This is sent to the feedback unit 28 which issues CPR feedback in the form of a ‘Start CPR and Push Hard’ instruction to the person using the CPR feedback system 20.

During a plurality of chest compressions by the person, the CPR assessment system 26 receives ECG signals from the ECG system 22, measured over a window of approximately 5 seconds to approximately 30 seconds. The CPR assessment system 26 uses the ECG signals to establish a current ECG signal metric, by establishing a current ECG score of the ECG signals. The current ECG score relates to quality of the measured ECG signals. The current ECG score is derived from one or more time-domain feature of the ECG signals and one or more frequency-domain features. The time-domain features include any of mean amplitude, peak amplitude, median slope of the ECG signals. The frequency-domain features include any of amplitude spectrum area (AMSA), power spectrum analysis features, centroid frequency. The time-domain features and the frequency-domain features may be used as measures to estimate subject condition.

During the plurality of chest compressions carried out by the person on the subject, the CPR assessment system 26 also receives oxygen signals from the oximeter of the biosignal system 24 and CPR chest compression depth signals from the accelerometer of the biosignal system 24. The oxygen signals and the CPR chest compression depth signals are measured over the same window of approximately 5 seconds to approximately 30 seconds. The CPR assessment system 26 uses the oxygen signals and the CPR chest compression depth signals to establish a current biosignal metric. In this embodiment, the current biosignal metric includes a current amplitude biosignal metric component for the oxygen signals of an average amplitude of the oxygen signals and a current amplitude biosignal metric component for the CPR chest compression depth signals of an average CPR chest compression depth.

The CPR assessment system 26 compares the current ECG signal metric with the reference ECG signal metric. This includes comparing the reference ECG score with the current ECG score. The CPR assessment system 26 further compares the current signal metric with the target signal metric. This includes comparing the target amplitude biosignal metric component of the oxygen signals with the current amplitude biosignal metric component of the oxygen signals and comparing the target amplitude biosignal metric component of the CPR chest compression depth signals with the current amplitude biosignal metric component of the CPR chest compression depth signals.

When the current ECG signal metric is less than the reference ECG signal metric (which suggests that the subject's condition is deteriorating) and the current biosignal metric is less than the target biosignal metric (which suggests that the CPR chest compressions are suboptimal), the CPR assessment system 26 produces a CPR feedback signal advising the person to improve CPR performance. The feedback signal is sent to the feedback unit 28 which issues CPR feedback in the form of a ‘Push Faster’, ‘Push Slower’ or ‘Push Harder’ instruction to the person using the CPR feedback system 20.

When the current ECG signal metric is less than the reference ECG signal metric (again suggesting that the subject's condition is deteriorating) and the current biosignal metric is equal to or greater than the target biosignal metric (which suggests that the target impedance signal metric may be improved), the CPR assessment system 26 increases the target biosignal metric and produces a CPR feedback signal advising the person to improve CPR performance. The feedback signal is sent to the feedback unit 28 which issues CPR feedback in the form of a ‘Push Harder’ instruction to the person using the CPR feedback system 20.

When the current ECG signal metric is equal to or greater than the reference ECG signal metric (suggesting that the subject's condition is improving), the CPR assessment system sets the reference ECG signal metric equal to the current ECG signal metric and produces a CPR feedback signal advising the person to maintain current CPR performance. The feedback signal is sent to the feedback unit 28 which issues CPR feedback in the form of a ‘Good Compressions’ instruction to the person using the CPR feedback system 20.

In this embodiment, the CPR assessment system 26 is configured to repeat some of the steps described above over a predetermined period of time. The pre-determined period of time is 2 minutes and is measured by a clock of the CPR assessment system 26. When the clock has not reached the pre-determined time the CPR assessment system 26 returns to the step of receiving ECG signals measured during a further plurality of chest compressions by the person. The further plurality of chest compressions do not overlap with the previous plurality of chest compressions. The analysis of the ECG and the biosignals, comprising oxygen signals and CPR chest compression depth signals, and feedback to the subject therefore continues over several pluralities of chest compressions during the 2 min time. When the clock reaches the pre-determined time, the CPR assessment system 26 produces a CPR feedback signal advising the person to stop CPR. The feedback signal is sent to the feedback unit 28 which issues CPR feedback in the form of a ‘Stop CPR’ instruction to the person using the CPR feedback system 20.

In this embodiment, the CPR assessment system 26 then returns to the step of establishing a reference ECG signal metric and a target biosignal metric and repeats the further steps as described above. This continues for as long as is deemed necessary.

Note that the CPR feedback signal described above numerous times as “a CPR feedback signal” can be the same signal or can represent different types of or different feedback signals. Thus, where a claim may suggest that it is the same signal, this disclosure includes the concept where the steps include producing a first CPR feedback signal advising the person to start CPR and when the current ECG signal metric is less than the reference ECG signal metric and the current biosignal metric is less than the target biosignal metric, producing a second CPR feedback signal advising the person to improve CPR performance and when the current ECG signal metric is less than the reference ECG signal metric and the current biosignal metric is equal to or greater than the target biosignal metric, increase the target biosignal metric and producing a third CPR feedback signal advising the person to improve CPR performance, and/or when the current ECG signal metric is equal to or greater than the reference ECG signal metric, set the reference ECG signal metric equal to the current ECG signal metric and producing a fourth CPR feedback signal advising the person to maintain current CPR performance. Several of these signals may be the same signal (such as the second and the fourth CPR feedback signal) but they also may be separate feedback signals.

Thus, the CPR feedback system 20 provides dynamic CPR feedback to the person performing multiple sets of CPR chest compressions on the subject, the CPR feedback being based on monitoring of the sets of chest compressions using biosignals comprising impedance signals, oxygen signals and CPR chest compression depth signals and monitoring of the ECG of the subject. It will be appreciated that other types of biosignals may be used.

In these embodiments, the CPR feedback system 20 is described as a stand-alone device. It will be appreciated that the CPR feedback system 20 may include a part of a further system, such as a defibrillator.

The system disclosed herein in any embodiment can include hardware components such as an input component, an output component, computer memory that is non-transitory such as RAM, ROM, a hard drive, firmware or other computer component, a bus, a computer processor, display such as a touch sensitive display, a casing, and so forth. The various computer components that can be used to provide the system or systems disclosed herein are part of this disclosure. Computer modules can include software that is programmed and stored in memory having instructions which, when executed by the processor, cause the processor to perform any one or more of the operations disclosed herein.

We claim:

1. A cardio pulmonary resuscitation (CPR) feedback system for assessing CPR carried out by a person on a subject and providing CPR feedback to the person, comprising:
an electrocardiogram (ECG) system configured to measure ECG signals of the subject;
a biosignal system configured to measure biosignals of the subject;
a CPR assessment system connected to the ECG system to receive ECG signals and connected to the biosignal system to receive biosignals; and
a feedback unit connected to the CPR assessment system and configured to receive CPR feedback signals and issue CPR feedback to the person,
wherein the CPR assessment system is configured to perform the steps:
(i) establish a reference ECG signal metric and a target biosignal metric;
(ii) produce a CPR feedback signal advising the person to start CPR;
(iii) receive ECG signals measured during a plurality of chest compressions and use the ECG signals to establish a current ECG signal metric;
(iv) receive biosignals measured during the plurality of chest compressions and use the biosignals to establish a current biosignal metric;
(v) compare the current ECG signal metric with the reference ECG signal metric and compare the current biosignal metric with the at least one target biosignal metric;
(vi) when the current ECG signal metric is less than the reference ECG signal metric and the current biosignal metric is less than the target biosignal metric, produce the CPR feedback signal advising the person to improve CPR performance;
(vii) when the current ECG signal metric is less than the reference ECG signal metric and the current biosignal metric is equal to or greater than the target biosignal metric, increase the target biosignal metric and produce the CPR feedback signal advising the person to improve CPR performance; and
(viii) when the current ECG signal metric is equal to or greater than the reference ECG signal metric, set the reference ECG signal metric equal to the current ECG signal metric and produce the CPR feedback signal advising the person to maintain current CPR performance.

2. A CPR feedback system according to claim 1 in which the biosignal system is configured to measure one or more type of biosignals of the subject.

3. A CPR feedback system according to claim 2, wherein the one or more type of biosignals comprise any of chest impedance signals, end-tidal carbon dioxide signals, saturation of peripheral oxygen signals, blood pressure signals, chest compression depth signals.

4. A CPR feedback system according to claim 1, wherein establishing the reference ECG signal metric comprises receiving ECG signals from the ECG system measured over a predefined period of time prior to commencement of CPR and using the ECG signals to establish the reference ECG signal metric.

5. A CPR feedback system according to claim 4 in which using the ECG signals to establish the reference ECG signal metric comprises establishing a score of an ECG signal derived from any of one or more time-domain features of the ECG signals, one or more frequency-domain features of the ECG signals, one or more time-domain features and one or more frequency-domain features.

6. A CPR feedback system according to claim 1, wherein establishing the target biosignal metric comprises receiving a pre-determined target biosignal metric comprising at least one target biosignal metric component for one or more types of biosignal comprising any of at least one target frequency biosignal metric component, at least one target amplitude biosignal metric component, at least one target frequency biosignal metric component and at least one target amplitude biosignal metric component.

7. A CPR feedback system according to claim 1, wherein establishing the target biosignal metric comprises (i) receiving one or more types of biosignals measured during a plurality of chest compressions by the person, and (ii) using the or each or some of the types of biosignals to establish the target biosignal metric.

8. A CPR feedback system according to claim 7, wherein the target biosignal metric comprises at least one target biosignal metric component for the or each or some of the types of biosignal comprising any of at least one target frequency biosignal metric component, at least one target amplitude biosignal metric component at least one target frequency biosignal metric component and at least one target amplitude biosignal metric component.

9. A CPR feedback system according to claim 1, wherein using the ECG signals to establish the current ECG signal metric comprises establishing a score of the ECG signals derived from any of one or more time-domain feature of the ECG signals, one or more frequency-domain features of the ECG signals, one or more time-domain features and one or more frequency-domain features.

10. A CPR feedback system according to claim 1, wherein using the biosignals measured during the plurality of chest compressions to establish the current biosignal metric comprises establishing at least one current biosignal metric component for at least one type of biosignals comprising any of at least one current frequency biosignal metric component, at least one current amplitude biosignal metric component, at least one current frequency biosignal metric component and at least one current amplitude biosignal metric component.

11. A CPR feedback system according to claim 10, wherein comparing the current biosignal metric with the target biosignal metric comprises comparing at least one current biosignal metric component for the at least one type of biosignals with at least one equivalent target biosignal metric component for the at least one type of biosignals.

12. A CPR assessment system according to claim 1, wherein any preceding claim configured to repeat steps (iii) to (viii) over a predetermined period of time and when the predetermined period of time has been reached, produce the CPR feedback signal advising the person to stop CPR.

13. A CPR assessment system according to claim 1, wherein the CPR assessment system is configured to repeat steps (i) to (viii).

14. A CPR assessment system according to claim 1, wherein the CPR assessment system is configured to carry out the steps:
produce the CPR feedback signal advising the person to perform CPR chest compressions at a rate equal to a rate of an audible metronome signal emitted by a metronome of the CPR assessment system,
receive ECG signals measured during the chest compressions and use the ECG signals to establish a current ECG signal metric,
compare the current ECG signal metric with the reference ECG signal metric,
when the current ECG signal metric is less than the reference ECG signal metric, adjust the rate of the audible metronome signal, produce the CPR feedback signal advising the person to adjust the rate of performance of the CPR chest compressions to equal an adjusted rate of the audible metronome signal and go back to the second step,
when the current ECG signal metric is equal to or greater than the reference ECG signal metric, produce the CPR feedback signal advising the person to maintain the rate of performance of the CPR chest compressions.

15. A CPR feedback system according to claim 1, wherein the CPR feedback system is part of a defibrillator.
